(19) United States
(12) Patent Application Publication
(10) Pub. No.: US 2009/0326359 A1
(43) Pub. Date: Dec. 31, 2009

(54) METHOD OF IN VIVO DETECTION AND/OR DIAGNOSIS OF CANCER USING FLUORESCENCE BASED DNA IMAGE CYTOMETRY

(75) Inventors: Bernardus Hendrikus Wilhelmus Hendriks, Eindhoven (NL); Ruth Wihelmine Ireen De Boer, Eindhoven (NL); Gerhard Spekowius, Eindhoven (NL); Stein Kuipeer, Eindhoven (NL); Nijs Cornelis Van Der Vaart, Eindhoven (NL)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONICS N.V., EINDHOVEN (NL)

(21) Appl. No.: 12/375,776

(22) PCT Filed: Jul. 11, 2007
(86) PCT No.: PCT/IB07/52764

§ 371 Date: Sep. 3, 2009

(30) Foreign Application Priority Data
Aug. 4, 2006 (EP) 06118437.0

(51) Int. Cl. A61B 6/00

(52) U.S. Cl. 600/407

(57) ABSTRACT

The invention relates to a method of determining in vivo in a human or animal subject the amount of nuclear DNA by first localizing cell nuclei of living tissue and subsequently measuring the nuclear UV absorbance using confocal scanning microscopy. The invention relates also to a method for detecting cancerous cells in vivo in a human or animal subject by first identifying the localization of cell nuclei in living tissue and subsequently determining the nuclear UV absorbance by laser scanning confocal microscopy. Furthermore, the invention relates to a method of diagnosing cancer in a human or animal subject in vivo relying on a combination of identifying the localization of cell nuclei in living tissue and measuring nuclear UV absorbance by laser scanning confocal microscopy.

The invention relates to a method of determining in vivo the amount of nuclear nucleic acids in cells of a human or animal subject by localization of the cell nuclei in vivo and measuring the nuclear absorption of ultra violet light.

The invention also concerns a method of detecting cancerous cells in vivo in a human or animal subject by localization of the cell nuclei in vivo and measuring the nuclear absorption of ultra violet light.

Similarly, the invention relates to a method of diagnosing and/or predicting cancer in a human or animal subject which is performed in vivo and which relies on the localization of cell nuclei and the measurement of nuclear UV absorption.

Early detection of cancer is a key feature in treating cancer patients. There are various possibilities of detecting cancerous cells in a biological sample and thus to diagnose an existing cancer or at least to estimate the likelihood of future cancer development. These different approaches comprise physical examination of cancer tissue, morphological characterization of cancerous cells, immunohistochemical staining and characterization of cellular structures such as membranes and the nucleus, measuring the expression of tumour specific factors, etc.

One possibility of detecting cancerous cells is so called DNA cytometry, which measures the amount of nuclear DNA in order to detect deviations from normal DNA content. It is assumed that if a cell as a consequence of mutagenic events comprises less or more DNA than a known standard, which is known to be of a non-cancerous, i.e. “healthy” or “normal” type, this deviation in DNA content will be indicative of major chromosomal rearrangements and thus ongoing cancer development.

Quantization of nuclear DNA by nucleic acids specific stains is thus increasingly coming into practice in both research and clinical applications in the context of cancer diagnosis.

The measurement of nuclear DNA content by either flow or image cytometry is based inter alia on the assumptions that (i) the amount of stain bound to DNA is proportional to the amount of DNA present and that (ii) the optical signal generated from the stain by emission, absorption or transmission is proportional to the amount of stain.

One DNA specific staining procedure which is typically used for the purpose of measuring nuclear DNA by DNA image cytometry is an acid-based reaction named after Feulgen and Rossenbeck, often simply referred to as the Feulgen reaction. Actually the Feulgen reaction is a chromogenic reaction in which DNA is hydrolyzed by an acid to create a purine-free DNA (i.e. so-called apurinic acid, APA) with free aldehyde groups. These are then reacted with a Schiff's reagent containing a dye that binds covalently to the free aldehyde groups.

While the Feulgen reaction is specific for DNA, it suffers from various drawbacks. It is a time-consuming and rather elaborate reaction that needs to be carefully controlled and validated in order to yield reproducible and meaningful results. One problem arises from the fact that the APA is hydrolyzed into smaller fragments by HCl which is commonly used for the removal of the purine bases. However, fragmentation of the APA molecules leads to removal of these fragments from the cell nucleus and thus to loss of stainable DNA material.

Besides these disadvantages that are inherent to the Feulgen staining method, the colouring of nuclear DNA by the Feulgen method is not allowed in vivo as the Feulgen staining itself may be mutagenic and thus give rise to the development of cancer.

Another key feature of prior art DNA image cytometry methods is that these methods require an operation mode in which cells are spread out over e.g. glass slides in order to allow measurement of individual cells. However, performing DNA cytometry measurements in vivo of course is complicated by the situation that one is not able to isolate specimens but rather has to perform the DNA measurement in a situation where it is not possible to isolate an individual cell from its physiological environment. Rather, when performing DNA image cytometry in vivo, i.e. in a human or animal subject, one will have to find a way to measure the amount of an individual cell without being able to separate it physically from its natural environment.

Attempts have been made in the art to allow for visualisation of individual cells in their natural environment by providing novel microscopic devices.

WO 2004/113962 A2 discloses a miniaturised confocal microscopic system that allows viewing cells within thick-layered specimens.

However, one of the prerequisites of DNA image cytometry in vivo, which has not been solved up to the present date, is that one determines the amount of nuclear DNA in the living tissue.

Thus, there is a continuing need for methods that allow a reliable, rapid and efficient way of measuring the amount of nuclear DNA and detection of cancerous cells and cancers in vivo in human or animal subjects.

It is an object of the present invention to provide a method for determining in vivo the amount of double stranded nuclear nucleic acid such as DNA in the nucleus of a cell.

It is also an object of the present invention to provide a method of detecting in vivo in a human or animal subject the presence of cancerous cells.

It is a further object of the present invention to provide a method that allows in vivo diagnosing of the presence of cancer or the likely development of cancer.

In order to achieve the above-defined objects, methods as defined in independent claim 1 are provided.

According to the invention, a method of determining in vivo the amount of nuclear nucleic acids in at least one cell of a human or animal subject is provided wherein said method comprises the steps of:
a) localization of the nucleus of said at least one cell in vivo in a human or animal subject;
b) measuring the absorption of ultraviolet (UV) light by the nucleus in vivo.

In one exemplary embodiment of the present invention, the aforementioned methods for determining in vivo the amount of nuclear nucleic acids within a cell may be used to detect at least one cancerous cell within a human or animal subject.

In a further embodiment of the aforementioned methods the nuclear UV absorbance obtained in step b) can be used to calculate the (aneu)ploidy state of the cell.

This can be done by referencing the obtained the nuclear UV absorbance to a nuclear UV absorbance obtained under substantially identical conditions for a cell for which one knows that it is of a non-cancerous type and for which the nuclear DNA content is known. In a preferred embodiment this calculation is done outside the human or animal body.

In yet another embodiment of the present invention, an aneuploidy state deviating by at least 10% from the values of 2 will be considered as indicative of a cancerous cell. In the case of a cell which in the healthy state would be proliferating a deviation of aneuploidy by at least 10% from the values of 2 and 4 will be considered to be indicative of ongoing cancer development.

In one embodiment of the present invention, methods of determining the amount of nuclear DNA within a cell as described above are used to detect at least one cancerous cell which is associated with a cancer selected from the group comprising leukemia, lymphoma, brain cancer, cerebrospinal cancer, bladder cancer, prostate cancer, breast cancer, cervical cancer, uterus cancer, ovarian cancer, kidney cancer, oral and throat cancer, esophageal cancer, lung cancer, colon rectal cancer, pancreatic cancer and melanoma.

In one of its embodiments, the method in accordance with the invention uses confocal laser scanning microscopy, two photon imaging, scanning optical coherence tomography, high resolution ultrasound or UV endomicroscopy for localization of the nucleus in step a) of the above described method.

In yet another embodiment of the present invention, measuring of nuclear UV light absorption in step b), is done by calibrating the UV light absorption signal and determining the volume of the nucleus.

In a preferred embodiment, determination of nuclear UV absorption is done by confocal laser scanning microscopy. A particularly preferred embodiment of the invention uses UV light for this purpose, which has a wavelength between approximately 240 nm to approximately 280 nm.

In another embodiment of the present invention, a method of in vivo diagnosing and/or predicting cancer in a human or animal subject is provided which comprises the steps of:
a) localization of the nucleus of said at least one cell in vivo in a human or animal subject;
b) measuring the absorption of ultraviolet light by the nucleus in vivo
c) determining the amount of nuclear nucleic acids by comparing UV; absorption of the nucleus as determined in step b) with UV absorption of a nucleus of a non-cancerous cell which has been obtained also using steps a) to b);
d) deciding on the presence or likely future occurrence of a cancer depending on the amount of nuclear nucleic acids.

In step c) the comparison can be made outside the human or animal body.

Yet another embodiment of this aspect of the invention relates to a method of diagnosing and/or predicting cancer in a human or animal subject with the method comprising the above-mentioned characteristics and wherein a ploidity state deviating by at least 10% from the values of 2 is indicative of a cancerous cell and thus on the presence or likely development of (future) cancer. In the case of a cell which in the healthy state would be proliferating a deviation of aneuploidy by at least 10% from the values of 2 and 4 will be considered to be indicative of ongoing cancer development.

In yet another embodiment of the present invention which is concerned with the method of diagnosing and/or predicting cancer in a human or animal subject of the afore-mentioned characteristics, the method is used to detect a cancer selected from the group comprising leukemia, lymphoma, brain cancer, cerebrospinal cancer, bladder cancer, prostate cancer, breast cancer, cervical cancer, uterus cancer, ovarian cancer, kidney cancer, oral and throat cancer, esophageal cancer, lung cancer, colon rectal cancer, pancreatic cancer, and melanoma.

In other embodiments of the present invention, relating to the diagnosis and prediction aspect, localization of the nucleus in step a) and measurement of nuclear UV light absorption in step b) may be performed as described above.

Thus, in a preferred embodiment, one will use confocal laser scanning microscopy to measure nuclear UV absorption, preferably with UV light of a wavelength between approximately 240 nm and approximately 280 nm.

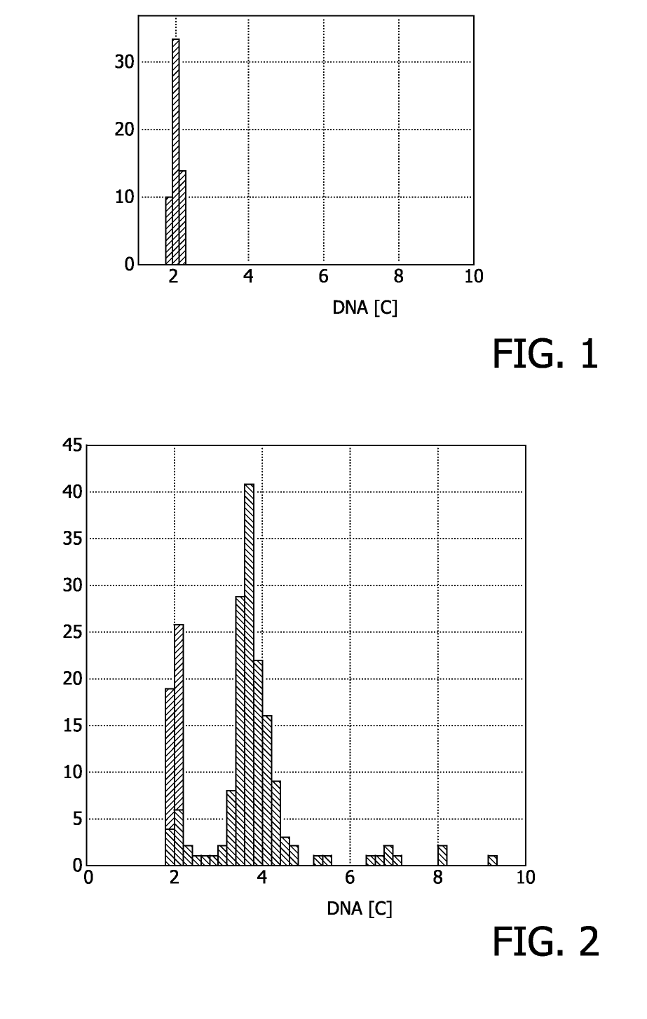

FIG. 1 shows a histogram as typically obtained if nuclear DNA content of a population of non-cancerous cells is determined. The cells are in a non-proliferative state.

FIG. 2 shows a histogram as typically obtained if nuclear DNA content of cancerous cells is determined. In this case the peak corresponding to 4 is indicative of cancer as cell are examined which are typically non-proliferative.

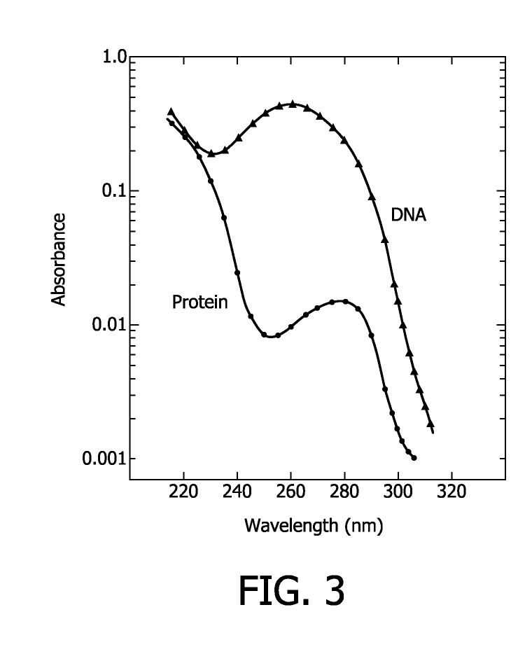

FIG. 3 shows the absorption factor of dissolved DNA and protein in the same concentration for the wavelength range of ultraviolet light.

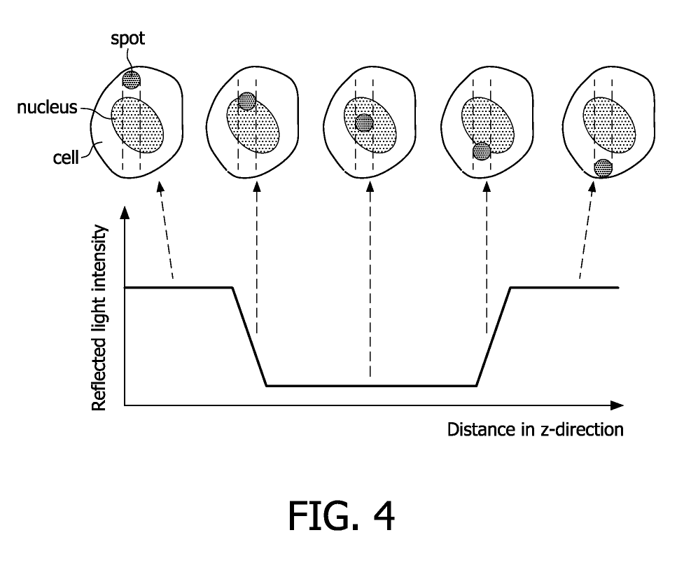

FIG. 4 shows schematically how the amount of reflected light and thus absorbance changes if a light spot of ultraviolet light is moved within one plane of a cell from the cytoplasm through the nucleus to other parts of the cytoplasm.

Identification of cancerous cells by DNA cytometry is typically carried out using flow cytometry or image cytometry.

In the case of DNA image cytometry which may also be designated as ICM, DNA is stained with a marker in order to visualize the DNA. Subsequently, alterations in the amount of the DNA which are commonly designated as aneuploidy are used to detect the presence of a cancerous cell as aneuploidy is considered to be characteristic of either an already existing cancerous state or a developing cancerous state. Thus, detection of DNA aneuploidy allows an early and sensitive diagnosis of cancer by detecting cancerous cells, often years ahead of morphological and histological characterization of tissue biopsies.

However, as mentioned above established DNA colouring reactions such as the Feulgen staining method are laborious and time-consuming. Furthermore, it is not possible to use DNA colouring reactions such as the Feulgen staining method in vivo as they are mutagenic in themselves and thus give rise to the development of cancer.

The inventors have surprisingly found that it is possible to determine the amount of nuclear nucleic acids of cells which are imbedded in their natural environment in vivo in a human or animal subject by first localising the nuclei of such cells and secondly measuring the absorption of ultraviolet light by the nucleus.

Thus, the present invention in one embodiment is directed to a method of determining in vivo in a human or animal subject the amount of nuclear nucleic acids in at least one cell comprising the steps of:
a) localisation of the nucleus of said at least one cell in vivo in a human or animal subject;
b) measuring the absorption of ultraviolet (UV) light by the nucleus in vivo.

Such methods can be used inter alia to determine the genome size of an individual or species. Of course, such methods can also be used to detect at least one cancerous cell within e.g. a living tissue.

For step b), using UV light of a wavelength of approximately 240 nm to approximately 280 nm is preferred. Particularly preferred is Particularly preferred may be UV light of a wavelength of 250 nm, 255 nm or 260 nm. The same applies for step a) if localisation of the nucleus is done using UV light. Thus, in one preferred embodiment one uses for steps a) and b) UV light of the aforementioned wavelength.

For the purposes of the present invention the term “cancerous cell” relates to any cell, cellular tissue or organ made of cells with some or all of the cells comprising an amount of nuclear DNA which deviates from the amount of nuclear DNA as determined for a non-cancerous cell as a consequence of chromosomal duplications, deletions, insertions, translocations, etc.

It is well known that insertions, duplications, deletions and translocations of chromosomal DNA are to a large extent responsible for the development of cancer and can thus be considered to be characteristic of cancerous cells.

Cells that can be investigated by the methods in accordance with the invention may be selected from the group comprising bone marrow cells, lymph node cells, lymphocytes, erythrocytes, neural cells, muscle cells, fibroblasts, keratinocytes, mucosal cells etc.

Similarly the methods in accordance with the invention can be used to analyse in vivo various tissues in the human or animal body. Such tissue may be part of organs such as the liver, the heart, the kidneys etc. and/or harbor the aforementioned cell types.

The above-specified method of determining in vivo in a human or animal subject the amount of nuclear nucleic acids, preferably for detection of at least one cancerous cell will now be described in more detail with respect to the single steps a) and b).

In a first step which has been designated above as step a), at least one cell within the human or animal subject will be analysed in vivo as regards the position of its nucleus. In a preferred embodiment, said at least one cell may be a cancerous or suspected to be cancerous, i.e. a putative cancerous cell.

Typically the localisation of the cell nuclei will be performed for cells in their natural environment, i.e. in living tissue. Localisation of the cell nuclei may be achieved, e.g. using confocal laser scanning microscopy, scanning optical coherence tomogramography, 2-photon imaging, high-resolution ultrasound or UV-endomicroscopy.

If the morphology of the tissue is known and hence the position of the nucleus, one can start measuring the amount of nuclear nucleic acids in each cell such as the DNA content (step b). While this may be preferred in one embodiment, the position and thus the localisation of the nucleus does not need to be determined exactly, but an approximation may be sufficient before measurement of e.g. nuclear DNA starts. Even knowledge of the cell boundaries may be sufficient. For this reason, it may not be necessary to use UV light for determining the localisation of the nucleus, even though this may be a preferred embodiment.

Confocal Laser Scanning Microscopy

One of the preferred embodiments of localising the nucleus of a cell in vivo relies on the use of confocal laser scanning microscopy, which in a further preferred embodiment may use UV light sources. Confocal microscopy offers several advantages over conventional optical microscopy, one of the most important being the elimination of out-of-focus information that distorts the image, controllable depth of field and sub-micron resolution. A further advantage of confocal microscopy is that fluorescence of various portions of the specimen that are out-of-focus can be filtered out and so do not interfere with the portions or sections that are in-focus thereby yielding an image that is considerably sharper and shows a better resolution than a comparable image obtained by classical light microscopy.

The basic principle of confocal scanning microscopy is the use of a screen with a pinhole at the focal point of the microscope lens system which is “conjugate” to the point at which the objective lens is focussed. Only light coming from the focal point of the objective is focussed at the pinhole and can pass through to the detector, which e.g. may be a charge couple device (CCD). Light coming from an out-of-focus section of the sample will be nearly completely filtered out.

Thus, a confocal microscope has a significantly better resolution than a conventional microscope for the x- and y-direction. Furthermore, it has a smaller depth of field in the z-direction. By scanning the focal point through the sample, it is thus possible to view different planes of a sample and to then rebuild a 3-dimensional image of the sample. Furthermore, confocal microscopy is compatible with different wavelengths of light.

If a confocal laser scanning microscope is integrated in e.g. an endoscopic device, an actuator may be used to scan the confocal microscope over the tissue of interest. A first coarse scan can then be used to determine the morphology of the tissue and the nuclei of interest may be identified from this screen. At this stage where one has localised the nucleus, one may then proceed to step b) for measuring the nuclear absorption of UV light.

For localisation of the nucleus the confocal scanning microscope may use monochromatic or polychromatic light however, monochromatic UV light with a wavelength between 240 nm and 280 nm may be preferred. Particularly preferred may be UV light of a wavelength of 250 nm, 255 nm or 260 nm.

As a confocal laser scanning microscope one may use a microscope such as LEICA DMLM and having a Qimaging Retiga 2000R FASTCooled Mono 12-bit camera unit (QImaging, Burnaby, BC, Canada) for measuring the signal intensity of the fluorescence signal. A Leica DM6000 may be particularly preferred.

For the purposes of the present invention where cells are to be viewed in their natural tissue environment, the confocal laser scanning microscope may be integrated into an endoscope. Systems, which are known for this purpose, differ mostly in the manner in which the image is scanned. Two rather advanced commercial systems are available, e.g. from Optiscan and Mauna Kir Technology. The Mauna Kir instrument is a proximally scanned system where the image is transferred down the endoscope with a coherent fibre optic bundle. A selected point or fibre is imaged into the sampled tissue at the distal end. This confocal endomicroscope may be delivered through the working channel of an endoscope. Since the field of view of the endomicroscope is small, placement of the probe is guided by standard video endoscopy. Hence, the endoscope platform must include both a video imager and the confocal microscope. Of course, the endoscope unit may also comprise or be coupled to computer devices and software packages that allow processing of the obtained images.

Detection units may e.g. be Optronics DEI-700 CE three-chip CCD camera connected via a BQ6000 frame-grabber board to computer. Alternatively one may use a Hitachi HV-C20 three-chip CCD camera. Software packages for image analysis may e.g. be the Bioquant True Color Windows 98 v3.50.6 image analysis software package (R&M Biometrics, Nashville, Tenn.) or Image-Pro Plus 3.0 image analysis software. Another system that may be used is the BioView Duet system (BioView Ltd, Rehovot, Israel which is based on a dual mode, fully automated microscope (Axioplan 2, Carl Zeiss, Jena, Germany), an XY motorized 8-slides stage (Marzhauser, Wetzler, Germany) a 3CCD progressive scan color camera (DXC9000, Sony, Tokyo, Japan) and a computer for control and analysis of the system and the data.

Optiscan has developed an endomicroscope employing distal scanning. In this system, a single fibre transmits light down the endoscope and the fibre end of the distal head is scanned spatially and imaged with optics into the tissue. Optiscan has co-developed with Pentax an endoscope with a confocal microscope integrated into the instrument, thereby freeing up the working channel. Images of tissues that are obtained using this system are of a resolution that allows identification of the localization of cell nuclei.

Other miniature confocal optical devices are described in WO 2004/113962 A2, which is hereby incorporated by reference. Systems for confocal imaging of living tissue are also described in WO 02/073246 A2 as well as in US 2005/0036667 both being also incorporated by reference.

Optical Coherence Tomography

Yet another possibility of localising cell nuclei in vivo in living tissue of a human or animal subject is the use of optical coherence tomography (OCT).

OCT is an imaging technology that achieves up to a few millimetres penetration depth (typically 1,5 bis 2 mm) at ultra high resolution (several microns) generating 3-D tissue images in real time. OCT provides 3-D structural images (tissue layers, density changes) for providing spectroscopic information and to achieve functional and molecular imaging at will. OCT is an interferometry-based technology which is capable of measuring signals as small as −90 dB. One standard fibre-based OCT set up is shown in FIG. 5.

A coupler splits light coming from a light source. While one arm serves as a reference arm of the interpherometer, the other one delivers light to the sample and is therefore called the sample arm. The scanning optics provide lateral-scanning capabilities so that the OCT set up obtains an axial scan (A-scan) for each lateral position. All A-scans combined form a 3-D structural image.

When obtaining each A-scan the reference mirror displacement provides depth information. It is also possible to obtain depth information by scanning wavelengths. Several more advance techniques have been developed to achieve higher depth information in shorter times. Spectroscopic OCT is the most advanced among those providing depth scans data without any moving parts.

Currently the fastest OCT system can produce images on around 30 frames per second with more than 1,000 A-scans per frame. Lateral resolution is only limited by the scanning optics and the light focussing system. Axial resolution is in turn light source dependent. A typical axial resolution currently accessible with commercial super luminescence diodes having a bandwidth around 70 nm at 930 nm is approximately 5 μm. One of the best-demonstrated resolutions of around 1.5 μm was achieved by using a Ti:sapphire fs laser.

For the purposes of the present invention, the above described OCT devices and particularly the spectroscopic OCT devices may be miniaturised so that they can be used i.e. in an endoscope system. For miniaturising OCT technology, one may e.g. refer to the proposals made in the publication in Optics Letters 29, 2261 (2004). Typically a miniaturised OCT device, which is capable of being used in an endoscopic system, will provide:
A light source. It determines axial resolution, which is proportional to the source bandwidth. Commercially available SLDs (super luminescence diodes) obtain around 5 μm axial resolution. Better resolution is available if Ti:Sapphire fs laser or Tungsten lamp (very low power) is used (resolution close to 1 μm). Tunable laser is needed for Fourier domain OCT.
A fiber optics components to provide light delivery, fiber coupler and/or circulator to realize Michelson interferometer.
A detection components. This will be dependent on the OCT type. Photodiodes will be typically used, but in the case of spectral OCT spectrally resolved detection will be needed (spectrum analyzer in combination with a liner CCD array).
In case of polarization or phase OCT the engine and the probe components have to be able to maintain light polarization properties.

High-Resolution Ultrasound

As mentioned above, localisation of the cell nuclei may also be performed using high-resolution ultrasound.

2-Photon Imaging

Of course, the cell nuclei may also be localised using 2-photon imaging technologies. The two-photon method allows real-time three-dimensional in-vivo imaging of tissue. The basic underlying principle is that in this technique, a fluorophore in the tissue is excited by the absorption of two photons of low energy, resulting in the emission of fluorescence. This opposes to conventional (confocal) microscopy, where a single higher-energy photon brings the fluorophore in excitation.

For the two-photon process to occur, a relatively high photon flux is needed, which is generally obtained in the focal point of a confocal microscope. As a consequence, advantages of confocal microscopy also apply to two-photon imaging, like three-dimensional imaging and a high resolution (˜0.2 micron lateral and 0.5 micron axial).

Due to the low energy of the excitation photons, single-photon absorption by the tissue is relatively low, which minimizes the amount of photo-induced damage in the tissue. This is a significant advantage for in-vivo applications. Moreover, the low absorption rate leads to a deeper penetration of the photons into the tissue, resulting in an imaging depth up to 0.5 mm.

In conclusion, two-photon imaging combines the advantages of high resolution confocal microscopy with a large imaging depth and a small amount of photo-induced damage. It allows real-time in-vivo tissue characterization down to cellular level and has been proved to be suitable for diagnosing diseases.

Turning to step b) of the above-described method, measuring the absorption of ultraviolet (UV) light by the nucleus in vivo may be achieved by different approaches.

Using a confocal microscope set-up one can focus a probe being inside the nucleus of the cell. Typically one will let the probe spot of the confocal set-up be smaller than the nucleus.

The amount of UV light reflected back from the nucleus as measured by the confocal set-up is inversely related to the amount of light absorbed by the area probed by the spot. Assuming that absorption is almost equal throughout the nucleus, the only thing left is to calibrate the measurement and to determine the size of the nucleus.

Calibration

Calibration consists of determining absorption of UV light by a nucleus and by the regions outside the nucleus within the same plane of the confocal scan. For both measurements in and outside the nucleus, the light has travelled almost through the same amount and distance of tissue before reaching the cell of interest resulting in comparable and similar background signals in both measurements. Therefore, the ratio between the measurements for nuclear absorptions versus non-nuclear absorption can be taken as an absolute measurement for absorption of the cell nucleus.

In a preferred embodiment one will use UV light of a wavelength between approximately 240 nm and approximately 280 nm. In a particularly preferred embodiment one will use UV light with a wavelength of approximately 250 nm, 255 nm or 260 nm. The term “approximately” in the context of wavelength relates to deviations of 1.5%, preferably 1% and most preferably of 0.5%. The reason for using UV light and particularly UV light of a wavelength between 240 nm and 280 nm is that DNA which is found in the nuclei of cells shows a much higher absorption in this wavelength range than for proteins of (see FIG. 3).

The nucleus, containing both proteins and DNA will therefore absorb more light than the rest of the cell, which contains mainly proteins, but rather no DNA.

Using preferably a confocal laser scanning microscope one can measure the reflection of UV light by material inside the nucleus and compare it to the reflection by the cell material outside the nucleus. By calculating the ratio of UV light absorbed by the nucleus to UV light absorbed by the regions outside the nucleus, one obtains a signal that is a measurement for the density of DNA within a single plane as obtained with a confocal scan.

Determination of Nucleus Volume

In the second step of determining the amount of nuclear nucleic acids within the nucleus, one has to determine the total volume of the cell nucleus. This can be done as follows.

By scanning a light spot in 3-D through the nucleus one can determine the intensity of the reflected light as described above when calibrating the measurement. The intensity of the reflected light will tell whether one is still inside the nucleus, has reached the boundary of the nucleus or whether it is already outside the nucleus. This is reflected in FIG. 4.

The signal of reflectable UV light differs when the microscope light spot is focussed inside the nucleus from when it is outside the nucleus as already explained above. When the focussed light spot is outside the nucleus, UV absorption is relatively low and a high intensity of reflected light is measured. When the focus of the light spot moves through the cell and passes the edge of the nucleus, the reflected light intensity gradually decreases because more light is absorbed in the nucleus. As long as the light spot is fully inside the nucleus, the reflected light signal is constant and comparatively low until the other edge of the nucleus is again reached. Then the signal will continuously increase to its former high value. The distance in the direction of movement over which the signal is comparatively low measures the local thickness of the nucleus.

As FIG. 4 illustrates for a single scan within a single plane of the nucleus, these 1-D scans can be extended to 3 dimensions to obtain the full shape of the nucleus by performing the same measurement for different planes. At different coordinates (xy) the local thickness of the nucleus is measured in the z-direction. Thus, by reconstructing a 3-D image from the different 1-D images of various planes one can reconstruct and calculate the appearance and volume of the nucleus.

Thus, measurement of nuclear UV absorbance is, according to the invention, obtainable by calibrating the UV absorbance signal in the nucleus and determination of the volume of the nucleus. For calibration, the ratio between UV light absorbance of regions within the nucleus to UV light absorbance for regions outside the nucleus is to be considered for a single plane.

Determination of the volume of the nucleus is then obtained by summing up the calibrated UV absorbance as obtained for each plane of the nucleus.

All this is preferably done using confocal laser scanning microscopy with UV light of a wavelength between approximately 240 nm and between approximately 280 nm. In a particularly preferred embodiment one will use UV light with a wavelength of approximately 250 nm, 255 nm or 260 nm.

Thus, by determining UV absorbance using confocal scanning microscopy for different planes of a nucleus, it is possible to determine the absorption of ultra violet light by a cell nucleus. As the amount of nuclear absorbed light and the amount of DNA is proportional, one can determine the amount of DNA in the nucleus as is required for DNA image cytometry.

Correlating the nuclear absorption signal with the amount of DNA within a cell nucleus can be done according to standard DNA image cytology procedures as described by the e.g. Hardie et al. (The Journal of Histochemistry and Cytochemistry (2002), 50 (6) (735-749), see also below)

Once one has determined and measured the nuclear absorption of ultra violet light and has repeated this process for several cells, one can e.g. calculate statistics on the amount of DNA contained in the various cells. From these statistics conclusions regarding the cancerous or non-cancerous state of the tissue can be drawn as is typically done in classical DNA image cytometry.

The person skilled in the art is of course well-acquainted with methods of recording UV absorbance signals as well as using these signals to calculate images of the e.g. cell nucleus.

Determination of signal intensities, i.e. the amount absorbed or transmitted light, will be done using instruments as typically used for these purposes.

Thus, one may e.g. use a charge-coupled device or a digital camera being linked to the microscope aperture that is used for viewing the cells when exposed to the above-mentioned light sources. One may of course also use films etc.

Fluorescence measurements can be made with any standard filter cube (consisting of a barrier filter, excitation filter and the dichroic mirror) or any customized filter cube for special applications provided that the emission spectrum is within the spectral range of the system sensitivity. Spectral bioimaging can also be used in conjunction with any standard spatial filtering methods such as dark field and phase-contrast and even with polarized light microscopy.

Detection units may e.g. be Optronics DEI-700 CE three-chip CCD camera connected via a BQ6000 frame-grabber board to computer. Alternatively one may use a Hitachi HV-C20 three-chip CCD camera. Software packages for image analysis may e.g. be the Bioquant True Color Windows 98 v3.50.6 image analysis software package (R&M Biometrics, Nashville, Tenn.) or Image-Pro Plus 3.0 image analysis software. Another system that may be used is the BioView Duet system (BioView Ltd, Rehovot, Israel which is based on a dual mode, fully automated microscope (Axioplan 2, Carl Zeiss, Jena, Germany), an XY motorized 8-slides stage (Marzhauser, Wetzler, Germany) a 3CCD progressive scan color camera (DXC9000, Sony, Tokyo, Japan) and a computer for control and analysis of the system and the data.

Before calculation of DNA content and correlating DNA content to the cancerous or non-cancerous state of a cell is described in more detail, further embodiments of the invention are described. These embodiments give different examples which technologies and approaches may be used to localise the nucleus in cells of living tissue and to measure absorption of ultra violet light by such nuclei.

In one embodiment, nuclear localisation as well as measuring nuclear UV absorption may be done using confocal laser scanning microscopy.

In this embodiment a confocal microscope will be integrated into e.g. an endoscopic device. The device furthermore has resemblance with an optical recording device. An actuator will be used to scan the confocal microscope over the tissue of interest. A first course scan is used to determine the morphology. From this scan, the nuclei of interest are identified. Subsequently, the nuclear UV absorption measurement and thus the DNA cytometry measurement on the nuclei are performed as described above.

The present invention in one embodiment thus also relates to the use of a confocal laser scanning microscopy optical device for determining in vivo the amount of nuclear nucleic acids in a human or animal subject. In a preferred embodiment the present invention relates to the use of a confocal laser scanning microscopy optical device for identifying in vivo cancerous cells in a human or animal being. The amount of nuclear nucleic acids may be determinded as described above by localizing the cell nucleus and measuring nuclear UV absorbance. The device can be a miniature confocal laser scanning microscopy device that is e.g. incorporated into a endoscopic device such as described in WO 02/073246, US 2005/0036667 and WO 2004/113962 A2.

In another embodiment, the morphology of the tissue may be determined by an endomicroscope which is a microscope being integrated at the tip of an endoscope. The endomicroscope in a preferred embodiment may operate with UV light. The endomicroscope such as one that is described in WO 02/073246 will be used to localise the cell nuclei. DNA cytometry measurement may then be performed using scanning confocal microscopy. An advantage of the addition of the endomicroscope is that the image of the morphology of the cells can be obtained in real time, as no scanning is required as for confocal microscopy.

The present invention in one embodiment thus also relates to the use of a device incorporating the equipment for an endomicroscope such as described in WO 02/073246 which may be operated with UV light and confocal laser scanning microscopy for determining in vivo the amount of nuclear nucleic acids in a human or animal subject. In a preferred embodiment the present invention relates to the use of such a device for identifying in vivo cancerous cells in a human or animal being. The amount of nuclear nucleic acids may be determined as described above by localizing the cell nucleus and measuring nuclear UV absorbance.

As a consequence of this real-time imaging, nucleus scanning with a confocal microscope for determining nuclear UV absorption can be performed highly accurately. Of course, the confocal microscope, which is used, for measuring nuclear UV absorption will have to be aligned with the endomicroscope in order to ensure that the same nuclei are measured.

In a third embodiment, the morphology and thus the localisation of the nucleus is determined by optical coherence tomography and scanning confocal microscopy does the nucleus probing and thus the measurement of nuclear UV absorption. If these two different approaches are used for localisation of the nuclei and determination of nuclear absorbance, the accuracy of DNA cytometry is increased.

The present invention in one embodiment thus also relates to the use of a device incorporating the equipment for optical coherence tomography and confocal laser scanning microscopy for determining in vivo the amount of nuclear nucleic acids in a human or animal subject. In a preferred embodiment the present invention relates to the use of such a device for identifying in vivo cancerous cells in a human or animal being. The amount of nuclear nucleic acids may be determined as described above by localizing the cell nucleus and measuring nuclear UV absorbance.

In a fourth embodiment, a transducer above 100 MHz uses high-resolution ultrasound to determine the localisation of nuclei within a tissue. Under these conditions it will be possible to obtain a resolution that can resolve cells in tissue. The nucleus scanning and measurement of nuclear UV absorption is again done with confocal laser scanning microscopy.

The present invention in one embodiment thus also relates to the use of a device incorporating the equipment for high resolution ultrasound application and confocal laser scanning microscopy for determining in vivo the amount of nuclear nucleic acids in a human or animal subject. In a preferred embodiment the present invention relates to the use of such a device for identifying in vivo cancerous cells in a human or animal being. The amount of nuclear nucleic acids may be determined as described above by localizing the cell nucleus and measuring nuclear UV absorbance.

In a fifth embodiment, the morphology and thus the localisation of the cell nuclei may be achieved by the methods described above, thus by e.g. laser scanning microscopy, by endomicroscopy, by optical coherence tomography and by high-resolution ultrasound. However, measuring the nuclear absorption of UV light will not be done using confocal scanning microscopy, but rather in a two-photon imaging process.

The present invention in one embodiment thus also relates to the use of a device incorporating the equipment for confocal laser scanning microscopy, endomicroscopy, optical coherence tomography and/or high resolution ultrasound and 2 photon imaging for determining in vivo the amount of nuclear nucleic acids in a human or animal subject. In a preferred embodiment the present invention relates to the use of such a device for identifying in vivo cancerous cells in a human or animal being. The amount of nuclear nucleic acids may be determined as described above by localizing the cell nucleus and measuring nuclear UV absorbance.

In case of two-photon imaging processes, the laser beam is brought into focus with a cell of interest. A detector module detects the fluorescence induced by the two-photon absorption. In such a case, one has to rely on the auto-fluorescence of DNA. One can, when using the two-photon imaging technology, implement in principle the same set-up as described for confocal laser scanning microscopy. However, as one is now looking for fluorescence produced by the two-photon absorption, one will place a filter in front of the detector to filter out any light having a different wavelength than that of the two-photon fluorescence. Using this approach, the accuracy of DNA cytometry may be increased.

In most of the afore-described embodiments, the first step of identifying the morphology of the investigated tissue and thus to localise the nuclei in the cells of said tissue has been achieved by performing a rather coarse scan followed by a refined scan with a confocal laser scanning microscope in order to measure nuclear UV absorption. However, it is also possible to perform only one high resolution scan in which confocal laser scanning microscopy is used to localise the cell nuclei and to measure the nuclear absorption.

The afore-described methods can be used for in vivo DNA-cytometry measurements in human or animal subjects in order to allow e.g. for in vivo real-time cancer detection. The method can thus be applied for example to detection of skin cancer, cancer of mucosal surfaces such as the oral cavity, the lungs, the larynx, the thyroid, and the uterine cervix. The methods can also be used to detect internal cancers if minimal invasive procedures such as endoscopic procedures are applied.

As the methods in accordance with the invention allow for real-time detection of cancerous cells, DNA image cytometry is significantly speeded up compared to the previously known methods which mainly rely on DNA image cytometry on isolated cell populations.

By only considering nuclear absorbance, i.e. UV light that is absorbed by cell nuclei, the method in accordance with the invention allows for a fast and accurate determination of the amount of nuclear nucleic acids within a cell nucleus as will be explained in the following.

It is common knowledge that most mammalian cells comprise a double set of each chromosome. Thus, the number of chromosomes may be calculated as 2n with n being the number of chromosomes. However, as cells replicate they double the number of chromosomes before separation into the daughter cells occurs.

During DNA replication and prior to cell division, mammalian cells will thus comprise a number of chromosomes, which can be calculated as 4n with n being again the number of chromosomes.

If the DNA amount of a mammalian cell is referenced to the number of chromosome, the DNA content of a non-cancerous cell which may also be designated as “healthy” or “normal” cell may thus be assigned the values 2 or 4 depending on the replication state of the cell.

The term “ploidy state” in the context of the present invention refers to the DNA content of a normal, non-cancerous cell and may have the values of 2 and 4 as explained above.

It is common knowledge in the art how to determine the ploidy state of a non-cancerous cell by e.g. using the aforementioned Feulgen staining method. In this context, the publication of Hardie et al. (The Journal of Histochemistry & Cytochemistry (2002), 50 (6), 735-749) is incorporated by reference as far as it describes the determination of DNA content of a cell using DNA staining methods.

In the section “Image Analysis Densitometry” on page 738 of the Hardie et al. reference it is explained how the signal intensities obtained by e.g. the Feulgen staining method or fluorescent markers can be converted into densitometric values. The same should apply for the absorption signals measured by the methods in accordance with the invention. As explained above a healthy, non-cancerous cell will comprise 2n or 4n chromosomes with n being the number of chromosomes. Thus, densitometric analysis of signal intensities as obtained from the Feulgen staining method or absorption signals as used in the present invention will lead to a densitometric profile with two peaks areas.

These two peak areas will be assigned the values of 2 or 4, respectively. An example of such a densitometric profile is provided in FIG. 1. In the specific case, a non-proliferating cell was analysed which explains why there is no peak corresponding to 4.

Briefly, for image analysis densitometry, the microscope field which one uses for viewing the cells and measuring the absorption signal is captured by a microscope-mounted CCD (charge coupled device) detection device or a digital camera, which are connected to a computer. The pictures being digitalized images are recorded as a series of pixels, each pixel being assigned a characteristic colour and intensity. The intensities are then typically converted by computer-based algorithms into absorbance values, which in turn are displayed by image analysis software as the aforementioned densitograms.

The person skilled in the art is, of course aware, that a meaningful and reliable densitogram of normal, non-cancerous cells should be preferably calculated from a population of numerous cells with a number of 25-100 cells typically being sufficient. In a preferred embodiment of the invention, the amount of nuclear DNA will thus be determined by measuring the nuclear UV absorbance as described above for approximately at least 30, 40, 50 or 60 cells.

Determining the nuclear DNA content or ploidy state of the putative cancerous cells using the above-described methods in accordance with the invention can achieve determination of the presence of cancerous cells. The determined ploidy state is then compared with the ploidy state of non-cancerous cells for which ploidy state has been determined from nuclear absorption intensities obtained by the identical method. In one embodiment of the invention, all these calculations may be done outside the human or animal body.

For determining of whether a putative cancerous cell is indeed a cancer-prone cell one will thus determine a densitogram as described above. The densitogram observed for non-cancerous cells will be designated as the “standard” or “reference” densitogram.

Preferably, such reference densitograms will be measured using the same method as for detecting the cancerous cells, but one will ensure that only non-cancerous cells are used. This may be achieved by using cells of the same individual but from other tissue sites than those that are suspected to be cancerous. Alternatively or additionally one may use identical or comparable cell types, from the same tissue for which morphology indicates a non-cancerous state. One should preferably use comparable cell types as long as it is ensured that these cells are non-cancerous. The number and type of cells to be studied in order to obtain a standard densitogram are well within the knowledge of the person skilled in the art and will vary between 25 and 100 cells and preferably around 30, 40, 50 or 60 cells.

For the purposes of the present invention, the term “comparable cell type”thus relates to a cell type that is of comparable origin and has comparable cellular characteristics as the suspected cancerous cell. If for example a mucosal cell will be tested for cancer development, the standard reference cell should also be of mucosal origin. If on the other side lymphoid cells are tested for cancer developments, the standard reference cell should be also of lymphoid origin in order to be a comparable cell type

The nuclear UV absorbance of the comparable cell type which is known to be non-cancerous and which is used as a standard reference for the nuclear UV absorbance obtained for the putative cancerous cell should be measured under highly similar if not identical conditions, if possible.

If in a putative cancerous tissue a sufficiently large number of cell nuclei is analysed, the “breadth” of the peaks corresponding to 2 and 4 and the occurrence of additional peaks may already be indicative of cancer development.

If for a certain cell, a ploidy state is determined which deviates from the aforementioned values of 2 or 4, this is indicative of either substantial duplications, insertions, deletions or chromosomal rearrangements and thus of cancerous cells. As in this case, the ploidy state of such a cell deviates from the values of 2 and 4, one also speaks of the aneuploidy state. This, of course, assumes that one considers a cell type which is typically proliferative even in its normal state.

Thus a ploidy value of 4 may be indicative of cancer development if one examines cells that are usually known to be non-proliferative.

If one examines cell which are known to be proliferative in their normal state a value of 4 will not be considered to indicative for cancer-development. In this case deviations from the values of 2 and/or 4 will be indicative for cancer development.

The term “aneuploidy state” in the context of the present invention thus relates to an abnormal amount of nuclear DNA within a cell.

Of course, nuclear UV absorbance and thus the densitogram of putatively cancerous cells will also be calculated from a population of cells and one typically will measure approximately 100 to 700 and preferably around 100 to 300 cells.

If one e.g. examines cancerous cells from liver tissue, one may obtain a densitogram with two main peaks, which are assigned the values of 2 and/or 4. However, other than in the case of non-cancerous cells the two main peaks densitogram will not be as sharp and narrow but rather broad. Additionally one will observe peaks and signals in the densitogram, which are below 2, above 2, below 4 and above 4. An example is provided in FIG. 2.

Since the majority of cells in any sample will have a DNA content of 2, deviation from the majority could also be considered as abnormal or suspicious. In the event of a cancerous tissue, where normal cells are in the minority, there will still be a large variance in the DNA content that can be used to label the sample as suspicious.

A cell will therefore be rated as cancerous if the densitogram that is calculated on the basis of the nuclear UV absorbance which are obtained in accordance with the invention as described above, shows peak signals outside the peaks corresponding to the values 2 (and 4 in case of cell which in its normal state is proliferative).

However, the peaks corresponding to the values of 2 (and 4) may themselves be indicative for cancerogenic potential of a cell, if they show a rather broad curvature. In order to decide whether a densitogram is indeed indicative for cancer development or not in view of the peak form corresponding to values 2 (and 4), one will superimpose the densitogram of a putative cancerous cell sample with a standard or reference densitogram as described above.

The areas under the curve (AUC) of the peaks of the densitograms being indicative of the values 2 and 4 of the standard densitogram are taken as being indicative of a non-cancerous cell and any deviations the AUCs of the corresponding peaks of the densitogram of the putative cancerous cell by at least 10% will be considered to be indicative of a cancerous cell or state. In a preferred embodiment the deviation will be at least 15%, at least 20%, at least 25%, at least 30%, at least 40% or at least 50%.

One may also apply the standards set forth in the publication of Haroske et. al. “1997 ESACP consensus report on diagnostic DNA image cytometry”, Analytical Cellular Pathology 17 (1998) 189-200 where it is explained in detail when a cell will be considered to be normal or cancerous.

Thus, a significant deviation of the nuclear UV absorbance of a suspected cancerous cell in comparison to the nuclear UV absorbance obtained for a comparable non-cancerous cell type is indicative of either ongoing or likely future cancer development.

For the purposes of the present invention, a significant aberration in DNA content will be considered to be indicative of cancer development if the ploidy state of the suspected cancerous cells deviates from the ploidy values of 2 (or 4) by at least 10%. In a preferred embodiment the deviation will be at least 15%, at least 20%, at least 25%, at least 30%, at least 40% or at least 50%. A peak in a densitogram will have assigned a value of 2 or 4 for this part of the peak that is identical with the corresponding peaks in a reference densitogram.

In accordance with established practice, one may designate the DNA content of a cell as peridiploid if the peak corresponding to peak value 2 of the reference densitogram ranges from 1.8 to 2.2.

The DNA content of a cell will be considered to be peritetraploid if the peak corresponding to peak value 4 of the reference densitogram ranges from 3.6 to 4.4.

Any values outside these ranges will be considered as X-ploid.

Cells or tissues with peripdiploid, peritetraploid and x-ploid values will considered as cancerous cells for the purposes of the present invention.

Thus, the above-described method can be used to calculate the ploidy (aneuploidy) state of a putative cancerous cell by referencing the nuclear UV absorbance as obtained in accordance with the method of the invention for the putative cancerous cell with a nuclear UV absorbance using the same approach for a comparable cell type which is known to be non-cancerous.

For the purposes of the present invention, the term “non-cancerous” refers to a cell that is known to show no indications of cancer development.

In one embodiment of the present invention, the cancerous cells may be associated with a cancer selected from the group comprising leukaemia, lymphoma, brain cancer, cerebrospinal cancer, bladder cancer, prostate cancer, breast cancer, cervical cancer, uterus cancer, ovarian cancer, kidney cancer, oral and throat cancer, oesophageal cancer, lung cancer, colon rectal cancer, pancreatic cancer, and melanoma.

In another embodiment of the present invention a method of diagnosing and/or predicting cancer in vivo in a human or animal subject is provided which comprises the steps of:
a) localization of the nucleus of said at least one cell in vivo in a human or animal subject;
b) measuring the absorption of ultraviolet light by the nucleus in vivo;
c) determining the amount of nuclear nucleic acids by comparing UV absorption of the nucleus as determined in step b) with UV absorption of a nucleus of a non-cancerous cell which has been obtained also using steps a) to b);
d) deciding on the presence or likely future occurrence of a cancer depending on the amount of nuclear nucleic acids.

The comparison in step c) may be done outside the human or animal body.

The steps a) to c) may be performed as described above for the method of determining in vivo in a human or animal subject the amount of nuclear nucleic acids.

Thus, for step b), using UV light of a wavelength of approximately 240 nm to approximately 280 nm is preferred. Particularly preferred is Particularly preferred may be UV light of a wavelength of 250 nm, 255 nm or 260 nm. The same applies for step a) if localisation of the nucleus is done using UV light. Thus, in one preferred embodiment one uses for steps a) and b) UV light of the aforementioned wavelength.

Of course, the explanations given above as to the determination of densitograms for the putative cancerous cell and reference or standard densitograms, the meaning of terms such as “cancerous cell”, “non-cancerous cell”, “ploidy”, “aneuploidy”, “peridiploid”, “peritetraploid”, “X-ploid”, “AUC” etc. equally apply in the context of the method of in vivo diagnosis and/or prevention of cancer in accordance with the invention.

In a last step which has been designated above as step d), one decides on the presence or likely future occurrence of cancer depending on the nuclear DNA content or ploidy state of the cells under examination.

A cancer diagnosis may be considered as positive if the nuclear DNA content or ploidy values as determined for the putative cancerous cells deviates by at least 10% from the nuclear DNA content or ploidy values of 2 (and 4) of a comparable cell type for which the nuclear UV absorbance has been measured under highly comparable of an identical condition and which is known to be non-cancerous. In a preferred embodiment the deviation will be at least 15%, at least 20%, at least 25%, at least 30%, at least 40% or at least 50%.

The areas under the curve (AUC) of the peaks being indicative of the values 2 (and 4) of a standard densitogram are taken as being indicative of a non-cancerous cell and any deviations the AUCs of the corresponding peaks of the densitogram of the putative cancerous cell by at least 10% will be considered to be indicative of a cancerous cell and thus will lead to a positive diagnosis. In a preferred embodiment the deviation will be at least 15%, at least 20%, at least 25%, at least 30%, at least 40% or at least 50%.

In accordance with established practice, one will designate the DNA content of a cell as peridiploid if the peak corresponding to peak value 2 of the reference densitogram ranges from 1.8 to 2.2.

The DNA content of a cell will be considered to be peritetraploid if the peak corresponding to peak value 4 of the reference densitogram ranges from 3.6 to 4.4.

Any values outside these ranges will be considered as X-ploid.

Any cell or tissue that has been rated as peridiploid, peritetraploid or X-ploid will be considered as cancerous and will lead to a positive diagnosis.

The method of diagnosing and/or predicting cancer may be used to diagnose and/or predict a cancer selected from the group comprising leukaemia, lymphoma, brain cancer, cerebrospinal cancer, bladder cancer, prostate cancer, breast cancer, cervical cancer, uterus cancer, ovarian cancer, kidney cancer, oral and throat cancer, oesophageal cancer, lung cancer, colon rectal cancer, pancreatic cancer, and melanoma.

1. Method of determining in vivo the amount of nuclear nucleic acids in at least one cell of a human or animal subject comprising the steps of:
a) localizing the nucleus of said at least one cell in vivo in a human or animal subject;
b) measuring the absorption of ultraviolet (UV) light by the nucleus in vivo.

2. Method according to claim 1,
wherein the method is used to detect at least one putative cancerous cell in a human or animal subject.

3. Method according to claim 2,
wherein the amount of nuclear nucleic acids is determined by comparing UV absorption of the nucleus as determined in step b) of claim 1 with UV absorption of a nucleus of at least one non-cancerous cell.

4. Method according to claim 3,
wherein a deviation in ploidy state or nuclear DNA content by at least 10% from the values of 2 is indicative of a cancerous cell.

5. Method according claim 3,
wherein a ploidy state or nuclear DNA content of 1.8 to 2.2 is indicative of a peridiploid state, a ploidy state or nuclear DNA content of 3.6 to 4.4 is indicative of a peritetraploid state and a ploidy state or nuclear DNA content outside these ranges is indicative of an X-ploid state.

6. Method according to claim 2,
wherein the at least one cancerous cell is associated with a cancer selected from the group comprising leukemia, lymphoma, brain cancer, cerebrospinal cancer, bladder cancer, prostate cancer, breast cancer, cervical cancer, uterus cancer, ovarian cancer, kidney cancer, oral and throat cancer, esophageal cancer, lung cancer, colon rectal cancer, pancreatic cancer, and melanoma.

7. Method according to claim 1,
wherein localization of the nucleus in step a) is done using confocal laser scanning microscopy, two-photon imaging, scanning optical coherence tomography, high-resolution ultrasound or UV endomicroscopy.

8. Method according to claim 1,
wherein measuring UV light absorption by the nucleus in step b) is done using confocal laser scanning microscopy.

9. Method according to claim 8,
wherein the UV light has a wavelength between approximately 240 nm and approximately 280 nm and preferably of 250 nm, 255 nm or 260 nm.

10. Use of a device for determining in vivo the amount of nuclear nucleic acids in at least one cell of a human or animal subject, wherein the device comprises the equipment for
a) confocal laser scanning microscopy, endomicroscopy, optical coherence tomography and/or high profile ultrasound application for performing step a) of claim 1; and
b) confocal laser scanning microscopy and/or two photon imaging for performing step b) of claim 1.

11. Use according to claim 10 with a device for performing both steps a) and b) comprising the equipment for confocal laser scanning microscopy.
